(19) United States
(12) Patent Application Publication
(10) Pub. No.: US 2017/0348491 A1
(43) Pub. Date: Dec. 7, 2017

(54) HANDHELD SPRING DRIVEN INJECTION DEVICE WITH FORCE REGULATION

(71) Applicant: Novo Nordisk A/S, Bagsvaerd (DK)

(72) Inventor: Ebbe Kiilerich, Copenhagen NV (DK)

(21) Appl. No.: 15/533,610

(22) PCT Filed: Jan. 6, 2016
(86) PCT No.: PCT/EP2016/050115

§ 371 Date: Jun. 6, 2017

(30) Foreign Application Priority Data
Jan. 8, 2015 (EP) 15150478.4

(51) Int. Cl. A61M 5/315; A61M 5/20; A61M 5/48; A61M 5/31

(52) U.S. Cl. CPC A61M 5/31553; A61M 5/31585; A61M 5/482; A61M 5/2033; A61M 2205/19; A61M 2205/583; A61M 2005/3126

(57) ABSTRACT

A handheld injection device (10) comprising an needle cannula, a reservoir configured for containing a liquid drug to be dispensed, a piston, a trigger button (22), an elongated, hollow outer housing (20) in which the reservoir is positioned, the needle cannula being in liquid communication with the reservoir and extending from one end of the housing, wherein the injection device further comprises a drive mechanism comprising a spring or a resilient member, the spring or a resilient member being configured to provide a driving force when the trigger button is pressed allowing the piston to be advanced by the drive mechanism thus causing a dose of the liquid drug to be dispensed, wherein the handheld injection device comprises a variable friction system being adapted to apply a variable counter force leveling out the driving force.

The present invention relates to a handheld injection device comprising a needle cannula, a reservoir configured for containing a liquid drug to be dispensed, a piston, a trigger button, an elongated, hollow outer housing in which the reservoir is positioned, the needle cannula is in liquid communication with the reservoir and extends from one end of the housing, wherein the injection device further comprises a drive mechanism comprising a spring or a resilient member, the spring or a resilient member being configured to provide a driving force when the trigger button is pressed, allowing the piston to be advanced by the drive mechanism thus causing a dose of the liquid drug to be dispensed.

### BACKGROUND OF THE INVENTION

Handheld drug injection devices, such as pen type drug delivery devices, are well known and may take many forms.

Generally, an injection device may comprise a reservoir, a cartridge, an ampoule or the like (forth only denoted reservoir) and an elongated hollow cylindrical housing in which the reservoir is attached or embedded. The reservoir is filled with a liquid drug to be dispensed by a user. A needle cannula is attached to the reservoir coaxially aligned, extending generally from one end of the housing. A drive unit for a piston within the reservoir is also arranged in the housing. The piston is advanced by a drive mechanism in the direction of the needle within the reservoir (often referred to as in the distal direction), thus causing a set dose of the liquid drug to be dispensed.

Pen type drug delivery devices including simple disposable devices that are little more than an ampoule with an injection mechanism or they may be durable devices adapted to be used with pre-filled cartridges. Regardless of their form and type, they have proven to be great aids in assisting patients to self-administer injectable liquid drugs and biological agents. They also greatly assist care givers in administering injectable medicines to those incapable of performing self-injections.

Pen type drug delivery devices have application where regular injection by persons without formal medical training occurs. This may be increasingly common among patients having diabetes, where self-treatment enables such patients to conduct effective management of their disease. In practice, such a drug delivery device allows a user to individually select and dispense a number of user variable doses of a medicament.

Some injection devices are configured for the user to manually displace the piston. Other types of injection devices may be operated by means of an energized resilient member in the device, and this stored energy is used to displace the piston during dispensing.

The energizing of the resilient member can be accomplished by the user during preparation of the device or it can be loaded when manufacturing the device.

However, conventional resilient members like springs and elastomers provide a non-constant force as it delivers its energy, which will results in a non-constant delivery flow rate, and since the rate and force of the injection varies, the speed of the injection will vary, which may be undesirable.

The patent application WO 2014/166891 discloses a dispensing speed control for use in an injection device and a handheld injection device comprising such a speed control mechanism. The speed control mechanism comprises friction means for retarding a first component part during dose dispensing depending on the position of the release button.

It is attractive to use a low cost conventional torsion spring for delivering a driving force (or rather a driving torque) for the injection device, especially for disposable devices. However, a conventional torsion spring provides a non-constant force as it delivers its energy, which will result in a non-constant flow rate of the selected dose, which may be undesirable for the user. Thus, the dispensing rate will decrease towards the end of the dispensing cycle.

In many cases, it is desirable to provide a handheld injection device which is inexpensive to manufacture but yet provide reliable and more level or consistent dispensing rate.

### BRIEF DESCRIPTION OF THE INVENTION

It is an object of the present invention to provide a handheld injection device which provides a more uniform dispensing of the contained liquid drug.

It is a further object of the invention to enable this while keeping low manufacturing costs.

This is achieved in that the handheld injection device comprises a variable friction system adapted to apply a variable counterforce leveling out the driving force.

Hence, in a first aspect of the invention a handheld injection device is provided comprising: a needle cannula, a reservoir configured for containing a liquid drug to be dispensed, a piston, a trigger button, an elongated, hollow outer housing in which the reservoir is positioned, the needle cannula being in liquid communication with the reservoir and extending from one end of the housing, wherein the injection device further comprises a drive mechanism comprising a spring or a resilient member, the spring or a resilient member being configured to provide a driving force when the trigger button is pressed, allowing the piston to be advanced by the drive mechanism thus causing a dose of the liquid drug to be dispensed, characterized in that the handheld injection device comprises a variable friction system being adapted to apply a variable counterforce leveling out the driving force.

Hereby is achieved that the variable friction system provides a variable counterforce which compensates for the non-constant driving force provided by the inexpensive torsion spring.

In an embodiment the drive mechanism comprises a torsion spring.

In an embodiment the variable friction system comprises a cylindrical scale drum comprising a thread groove and an arm, the arm being configured to engage the thread groove to cause a variable friction.

In an embodiment aimed for leveling out the force from a factory loaded, fully energized spring the variable friction system comprises a piston rod and one or more guiding nuts, the piston rod comprising a varying cross-sectional diameter, having a largest diameter at the distal end, the one or more guiding nuts having complementary shaped surfaces configured for gradually decreasing the variable friction, whereby the friction loss is reduced when the piston rod is moved past the one or more nuts, during dispensing of liquid.

In an embodiment the thread groove comprises a gradually varying width.

In an embodiment the width of the thread groove increases during dispensing of liquid, whereby the variable friction between the thread groove and the arm decreases during liquid dispensing.

In an embodiment the arm comprises two prongs, the two prongs being adapted to engage with the thread groove. Hereby is achieved that the arm may more easily engage with the thread groove due to the flexibility of the prongs.

In an embodiment the trigger button is structurally connected to a flange, the flange comprising an abutment surface which is configured to be positioned adjacent to the arm without affecting the position of the arm in a non-active injection state of the handheld injection device.

In an embodiment the arm comprises a contact surface and the flange comprises an inclined contact surface, the inclined contact surface being configured to be positioned adjacent the contact surface of the arm, in a liquid drug dispensing state.

In an embodiment the handheld injection device comprises a first friction setting element configured for increasing or decreasing the level of variable counterforce.

In an embodiment the first friction setting element comprises an inclined contact surface adapted to provide a variable pressure to the flange.

In an embodiment the first friction element is movable in a direction tangential to the periphery of the outer housing.

In an embodiment the arm comprises an elongated portion extending along the length of the handheld injection device, the one end of the elongated portion being attached to a part of the outer housing and the other distal end of the elongated portion of the arm being adapted to engage with the thread groove.

In an embodiment the handheld injection device comprises a second friction setting element, positioned moveably along the elongated portion of the arm, the second friction setting element being configured to change the distance between the attachment point of the arm and the distal end of the arm.

In an embodiment the outer housing comprises a hollow space beneath the inner surface adapted to allow deflection of the elongated portion of the arm.

Various types of injection devices exist in the marketplace today. For example, some injection devices have a pre-mounted needle cannula, some are prepared for receiving a separate needle cannula, e.g. provided as part of a needle assembly, and some are needle-free and offer high pressure dose expelling through a jet nozzle. Also, some injection devices have a pre-mounted, non-exchangeable drug dedicated reservoir, while others are configured to receive and retain an exchangeable drug dedicated reservoir. A person of skill in the art will immediately realise that whether the drug is expelled through a needle cannula, pre-mounted or user mounted, or a jet nozzle, and whether the injection device has a pre-mounted or user attached reservoir is irrelevant to the practice of the present invention.

In a second aspect of the invention a handheld injection device is provided comprising a housing extending along a central axis and having a window, a dose setting mechanism comprising a dose setting member operable to set a dose of drug to be expelled from a reservoir, and a scale drum carrying a plurality of dose indicating indicia, a dose delivery mechanism comprising an expelling structure for expelling a set dose of drug from the reservoir, a spring member for providing energy to actuate the expelling structure, and a trigger button operable to cause energy to be released from the spring member, and a variable friction system comprising a contact member adapted to mechanically interface with the scale drum. The scale drum is configured to move relative to the window to a dose set position in response to a dose setting operation of the dose setting member and to move from the dose set position to a dose expelled position in response to a dose expelling operation of the trigger button, and the variable friction system is configured to provide a friction force between the contact member and the scale drum which decreases gradually as the scale drum moves from the dose set position to the dose expelled position.

The injection device is thus preferably of the type which offers setting and delivery of a plurality of different doses, respectively defined by a unique position of the scale drum relative to the housing. In such type of injection device the scale drum typically alternates between moving in one direction to set a dose and moving reversely during delivery of the set dose. Hence, each time a dose is delivered the scale drum undergoes one cycle of moving to a selectable dose set position and moving back to the dose expelled position. These movements are well-defined and a variable friction system based thereon thus enables the provision of a both reliable and reproducible counter-force to compensate for the reduction in the spring member output during the dose expelling, such that a constant force profile of the expelling structure is expressed.

In particular embodiments of the invention the scale drum comprises an exterior groove having a width and a depth, the contact member is configured to travel the exterior groove from a first position defined by the dose set position to a second position defined by the dose expelled position during expelling of the set dose, and the contact force between the contact member and the exterior groove decreases gradually as the contact member travels from the first position to the second position.

The exterior groove may extend helically along an exterior surface of the scale drum, and the contact member may be axially fixed with respect to the housing during expelling of the set dose. The contact member thus travels the exterior groove when the scale drum moves helically within the housing from the dose set position to the dose expelled position. The housing may comprise an interior protrusion configured for engagement with the exterior groove to effect the helical movement of the scale drum.

In some embodiments of the invention the width of the exterior groove increases gradually from the first position to the second position. Thereby, the friction force between the contact member and the exterior groove decreases gradually during expelling of the set dose because the engagement between the two is loosened as the contact member travels from the first position to the second position.

In other embodiments of the invention the depth of the exterior groove increases gradually from the first position to the second position. If the radial position of the contact member within the housing is constant during expelling of the set dose the friction force between the contact member and the exterior groove will decrease gradually as the contact member travels from the first position to the second position.

In particular embodiments of the invention the contact member comprises two prongs adapted to engage with the exterior groove.

The contact member may form part of the housing or, alternatively, form part of an insert which is axially fixed with respect to the housing. Particularly, the contact member may be arranged on a radially deflectable portion, of the housing or of the insert, such that the contact member is capable of relative radial motion but prevented from relative axial motion (disregarding any axial displacement component arising from a radial displacement) with respect to the housing. This will allow for a variable contact force between the contact member and the scale drum at any specific point along the exterior groove, even for a disengagement of the contact member from the exterior groove.

The contact member may be operatively coupled with the trigger button, and the variable friction system may be configured to switch from a disengaged state in which the contact member is disengaged from the scale drum to an engaged state in which the contact member is engaged with the scale drum in response to the dose expelling operation of the trigger button, and to switch from the engaged state to the disengaged state in response to a subsequent operation of the trigger button.

The trigger button may be movable, e.g. axially, relative to the housing between an idle position in which the dose setting member is operable to set the dose and an activated position in which the expelling structure is actuated to expel the set dose, and the dose expelling operation of the trigger button may comprise moving the trigger button from the idle position to the activated position, and the subsequent operation of the trigger button may comprise moving the trigger button from the activated position to the idle position.

In this case the variable friction system is only active when a dose is being expelled, i.e. it is not active when a dose is being set. The user will therefore not experience an increasing degree of resistance during dose setting as the dose is increased.

The trigger button may be biased towards the idle position, e.g. by a spring or a foam pad or the like, whereby the subsequent operation of the trigger button may simply comprise removing a depressive force therefrom.

In particular embodiments of the invention the trigger button is structurally connected to a flange comprising an abutment surface configured to interact with the radially deflectable portion of the housing and cause a radially inward deflection of the contact member towards the central axis when the trigger button is moved from the idle position to the activated position.

The abutment surface may comprise an inclined portion causing the radially inward deflection of the contact member to vary as a function of the axial position of the trigger button relative to the housing. It is thereby possible for the user, once the spring member has been released, to control the rate of the dose expelling by depressing the trigger button more or less against the housing, thereby altering the level of the contact force between the contact member and the scale drum.

In the present specification, reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in, or inherent of, at least that one aspect or embodiment of the invention, but not necessarily in/of all aspects or embodiments of the invention. It is emphasized, however, that any combination of the various features, structures and/or characteristics described in relation to the invention is encompassed by the invention unless expressly stated herein or clearly contradicted by context.

The use of any and all examples, or exemplary language (e.g., such as, etc.), in the text is intended to merely illuminate the invention and does not pose a limitation on the scope of the same, unless otherwise claimed. Further, no language or wording in the specification should be construed as indicating any non-claimed element as essential to the practice of the invention.

### BRIEF DESCRIPTION OF THE DRAWINGS

Embodiments of the invention will be described in the following with reference to the drawings wherein

FIG. 1 is a schematic side view of one embodiment of a variable friction system comprising a dose scale drum and a trigger button, shown in a non-activated and in an operative state, respectively;

FIG. 2 is a schematic side view of another embodiment of a variable friction system comprising a dose scale drum and a trigger button, one view where the variable friction system is non-activated and two views in an operative state, respectively;

FIG. 3 is a schematic side view of yet another embodiment of a variable friction system comprising a friction setting element;

FIG. 4 is a longitudinal partial cross-sectional side view of an embodiment of the variable friction system in a non-active state and a perspective view of the side view;

FIG. 5 is a longitudinal partial cross-sectional side view of the variable friction system of FIG. 4 in a dose-setting state and a perspective view of the side view;

FIG. 6 is a longitudinal partial cross-sectional side view of the variable friction system of FIGS. 4 and 5 in a dose-setting state, medium speed and a perspective view of the side view;

FIG. 7 is a longitudinal partial cross-sectional side view of the variable friction system in a dispensing-state and a perspective view of the side view;

FIG. 8 is a longitudinal partial cross-sectional side view of another embodiment of a variable friction system in a non-active state and a perspective view of the side view;

FIG. 9 is a schematic side view of another embodiment of a variable friction system comprising a piston rod.

### DETAILED DESCRIPTION OF THE INVENTION WITH REFERENCE TO THE FIGURES

The present invention relates to a handheld injection device (10).

Various aspects and embodiments of a handheld injection device for delivering set doses of a liquid drug as disclosed herein will now be described with reference to the figures.

When relative expressions such as “upper” and “lower”, “clockwise” and “counter clockwise” or similar are used in the following terms, these only refer to the appended figures and not to an actual situation of use. The shown figures are schematic representations for which reason the configuration of the different structures as well as their relative dimensions are intended to serve illustrative purposes only.

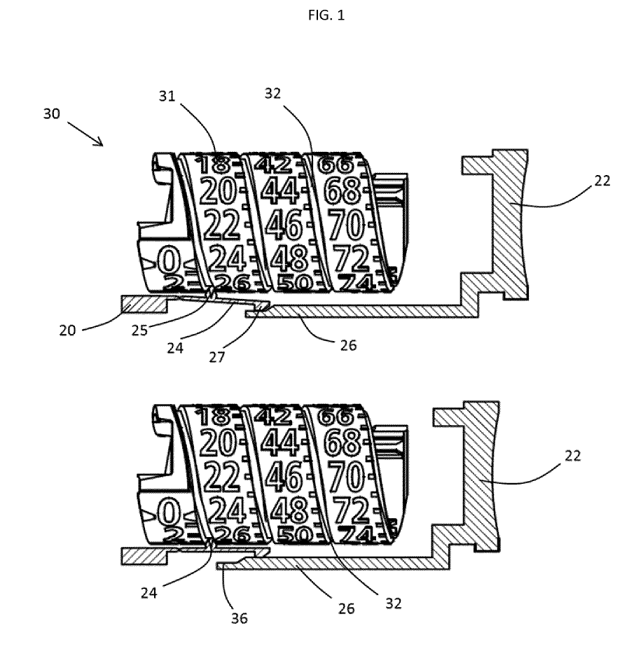

In that context it may be convenient to define that the term “distal end” in the appended figures is meant to refer to the end of the injection device which usually carries the needle cannula and as depicted e.g. in FIG. 1 whereas the term “proximal end” is meant to refer to the opposite end pointing away from the needle cannula.

Some of the different components are only disclosed in relation to a single embodiment of the invention but is meant to be included in the other embodiments without further explanation.

Generally, an injection device may comprise a reservoir and an elongated hollow cylindrical housing in which the reservoir is attached or embedded. The reservoir is filled with a liquid drug to be dispensed by a user. A needle cannula is attached to the reservoir coaxially aligned, extending generally from one end of the housing. A drive unit for a piston within the reservoir is also arranged in the housing. The piston is advanced by a drive mechanism in the direction of the distal end within the reservoir, thus causing a set dose of the liquid drug to be dispensed.

The driving force provided by the drive mechanism comprises a spring or other type of resilient member, such as a torsion spring. This drive mechanism may be energized by the user prior to every dose setting or the drive mechanism may be energized from factory and holds the energy to expel the full content of the reservoir. When the liquid drug within the reservoir is dispensed, the driving force provided by the torsion spring will decrease during dispensing the fluid. To compensate for the non-constant force as the torsion spring delivers its energy, and the corresponding non-constant flow rate of the selected dose, a variable counterforce system is provided which compensates for the non-constant driving force provided by the drive mechanism.

There are basically two types of drug delivery devices, resettable devices (ie. reuseable) and non-resettable (ie. disposable). Disposable devices need not have a resettable dose setting mechanism.

FIG. 1 schematically illustrates a partial longitudinal section of a handheld injection device in a non-active state and in an active dispensing state, respectively.

The top illustration shows a cylindrical scale drum (30) comprising indication (31) of a dose scale and a helix thread groove (32). The helix thread groove comprises a varying width, increasing gradually towards the distal end. An arm (24) is attached to an outer housing (20) of the handheld injection device. The arm comprises a contact surface (27) and two prongs (25). A trigger button (22) is in structural connection with a flange (26) and the contact surface (27) of the arm (24) is positioned adjacent an abutment surface of the flange (26) and the cylindrical scale drum.

The value of a desired dose is set by turning the scale drum one way (in the shown and corresponding embodiments, clockwise around the center axis seen from the trigger button). In this non-active state of the injecting device, the arm (24), which in this particular and corresponding embodiments, comprises two prongs (25) does not engage with the groove (32), whereby setting the dose by turning the scale drum is not or only minimally influenced by a friction force applied by the arm (24) and the two prongs (25).

The scale drum (30) and a drive spring may be structurally connected, such that during dose setting in the preparation state the drive spring may by winded up and tensioned, thereby preparing the drive mechanism for dispensing when the trigger button is pressed and the drive mechanism is activated.

Generally, in some embodiments, the scale drum may avoid variable friction when setting the dose but provide friction when the dose is given.

In the bottom view the injection device is illustrated in an activated state. The trigger button (22) is pressed partly inwards towards the scale drum simultaneously activating the drive mechanism (not shown), releasing force for dispensing fluid drug, and moving the flange (26), such that the contact surface (27) of the arm (24) is positioned abutting the inner surface of the flange (26) due to the thickness of the flange. The two prongs (25), which in this stage are pressed into the thread groove (32) by the flange (26), apply a variable friction due to the varying width of the thread groove (30). The width of the thread groove is increasing gradually towards the distal end. The variable friction applied to the injection device provides a variable counterforce, resulting in an at least more uniform dispensing force.

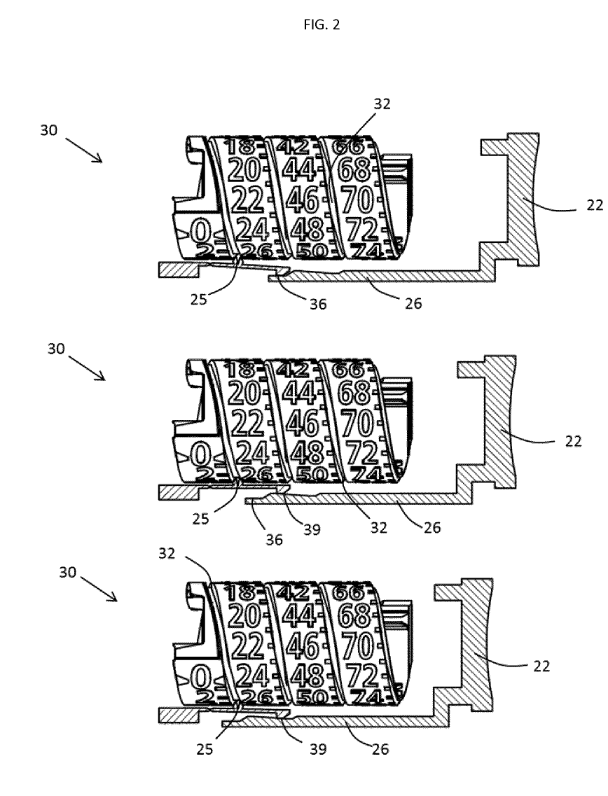

FIG. 2 schematically illustrates a partial longitudinal section of a handheld injection device in a non-active state and subsequently in two active dispensing states, respectively.

The top illustration shows the cylindrical scale drum (30) comprising indication of the dose scale and the helix thread groove (32). The helix thread groove comprises a varying width which decreases towards the proximal end. The trigger button (22) is in structural connection with a flange (26). The arm comprising the two prongs (25) are positioned adjacent an abutment surface (36) of the flange (26), in shape of an indent, and the cylindrical scale drum. In this non-active dispensing state, where the arm is not activated, the scale drum (30) may be rotated clockwise around the center axis of the injection device (seen from the trigger button) to set the dose to be dispensed.

In the middle illustration the trigger button (22) is pressed partly inwards towards the scale drum simultaneously activating the drive mechanism (not shown), releasing the spring force for dispensing, and the flange (26) is moved, such that the contact surface (27) of the arm is positioned abutting an inclined contact surface (39) of the flange (26). In this active dispensing state the scale drum (30) rotates counter clockwise around the center axis of the injection device (seen from the trigger button). The winded spring emits the stored energy and the resilient arm will pass the vertex of the flange and abut the inclined contact surface (39). The two prongs (25), which are pressed into the thread groove by the flange (26) apply a variable friction, due to the varying width of the thread groove, thus providing a variable counter force, resulting in an at least more uniform dispensing force.

Additionally, the user is able to adjust the level of friction and thereby the flow rate by the pressure applied to the trigger button, as the corresponding inclined contact surface (39) of the flange allows the user to adjust the friction force between the two prongs (25) and the thread groove (32) due to its inclination, during dispensing of the liquid drug.

The bottom view illustrates the trigger button pressed further and the inclined contact surface (39) applies less pressure on the arm (24).

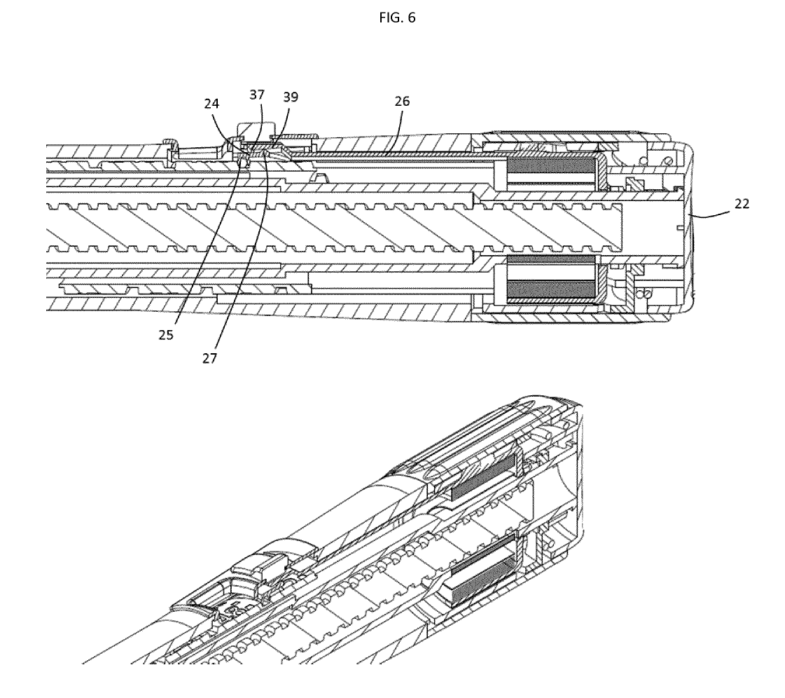

The inclined contact surface (39) of the flange has more thickness near the distal end of the flange, which presses the arm and the two prongs (25) towards the thread groove (32) of the scale drum (30) to cause increased friction. When the trigger button is pressed maximally, the contact surface of the arm is positioned where the inclined contact surface (39) provide most room and thus less pressure on the contact surface (27) of the arm. Thereby, the friction between the two prongs (25) and the thread groove (32) is decreased. Thereby, the user may be able to speed up the dispensing period during dispensing. This active dispensing state is illustrated in the bottom view of FIG. 2. FIG. 6 illustrates a similar active state.

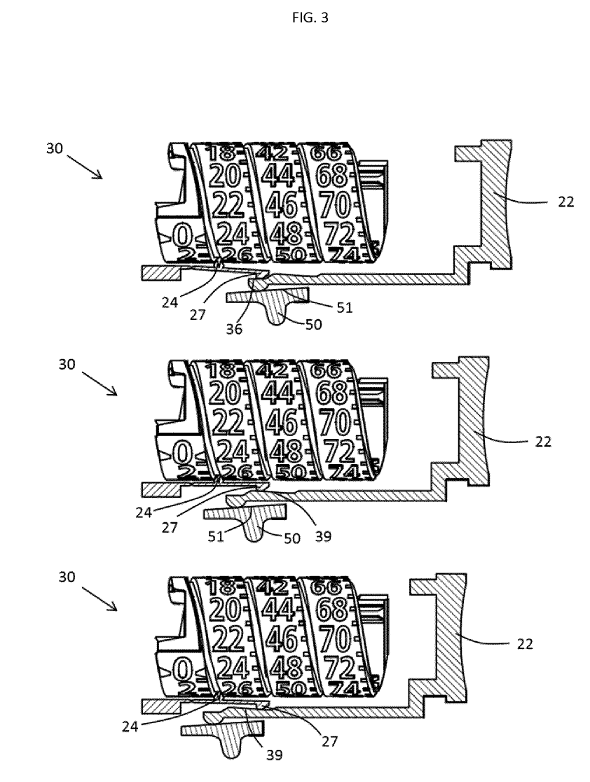

FIG. 3 illustrates a further embodiment of the handheld injection device (10) as illustrated in FIG. 2.

The schematic top view in FIG. 3 shows the cylindrical scale drum (30) comprising the helix thread groove. The helix thread groove comprises a varying width which is gradually increasing towards the distal end of the handheld injection device.

In this non-active dispensing state, where the arm (24) is not activated yet, the scale drum (30) may be rotated clockwise around the center axis of the injection device (seen from the trigger button) to set the dose to be dispensed. During this action the torsion spring may be winded up by the user.

Generally, the torsion spring may be winded up by the user prior to every dose setting or the spring can be winded during production, and hold the energy to expel the full content of the reservoir.

The handheld injection device comprises a first friction setting element (50) which comprises an inclined contact surface (51) adapted to provide a variable pressure to the flange (26). This first friction setting element may be set prior to dispensing. The first friction setting element (50) is positioned as to set the start level of variable counterforce.

In the middle illustration the trigger button (22) is pressed partly inwards towards the scale drum simultaneously activating the drive mechanism and releasing the spring force for dispensing, and the flange (26) has be moved such that the contact surface (27) of the arm (24) is positioned abutting an inclined contact surface (39) of the flange (26). The first friction setting element (50) is positioned so as to set a start level of variable counterforce, as the first friction setting element (50) increases or decreases the level of friction between the arm (24) and the thread groove.

In this active dispensing state the scale drum (30) rotates counter clockwise around the center axis of the injection device (seen from the trigger button). The winded spring emits the stored energy and the resilient arm (24) will pass the vertex of the flange (26) and abut the inclined contact surface (39). The two prongs, which are pressed into the thread groove by the flange (26), apply a variable friction due to the varying width of the thread groove, thus providing a variable counter force, resulting in a more uniform dispensing force.

Additionally, the user is able to adjust the speed during dispensing by the pressure applied to the trigger button, as the corresponding inclined contact surface (39) of the flange allows the user to adjust the friction force between the two prongs and the thread groove.

The bottom view illustrates the trigger button (22) pressed further and the inclined contact surface (39) applies less pressure on the arm (24).

The inclined contact surface (39) of the flange has more thickness near the distal end of the flange, which presses the arm (24) and the two prongs towards the thread groove of the scale drum (30) to cause increased friction. When the trigger button is pressed maximum, the contact surface of the arm is positioned where the inclined contact surface (39) provide most room and thus less pressure on the contact surface (27) of the arm. Thereby, the friction between the two prongs and the thread groove is decreased. Thereby, the user may be able to speed up the dispensing period.

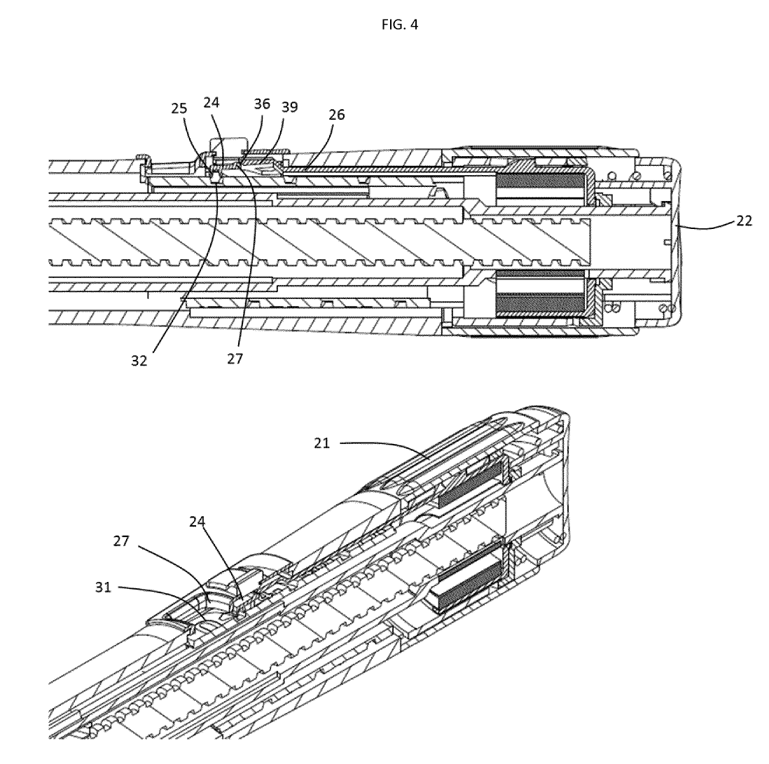

FIG. 4 illustrates an embodiment of the handheld injection device in a non-operative state. The injection device comprises a piston rod configured by the drive mechanism to be advanced in the direction of the needle within the reservoir, thus causing a set liquid drug dose to be dispensed.

The trigger button (22) is in structural connection with the flange (26). The arm (24) is positioned adjacent the abutment surface (36) of the flange (26) and the cylindrical scale drum. In this non-active state the arm is positioned adjacent and does not engage with the groove (32) of the scale drum. In the figure the scale drum and the digit indicating a value is visible thought the opening (27) in the outer housing. In this state the dose value is “0”, and the scale drum is located towards the trigger button.

The dose setting knob (21) is used to set the value of the desired dose. By turning the dose setting knob, the scale drum may be turned one way and a torsion spring is tensioned.

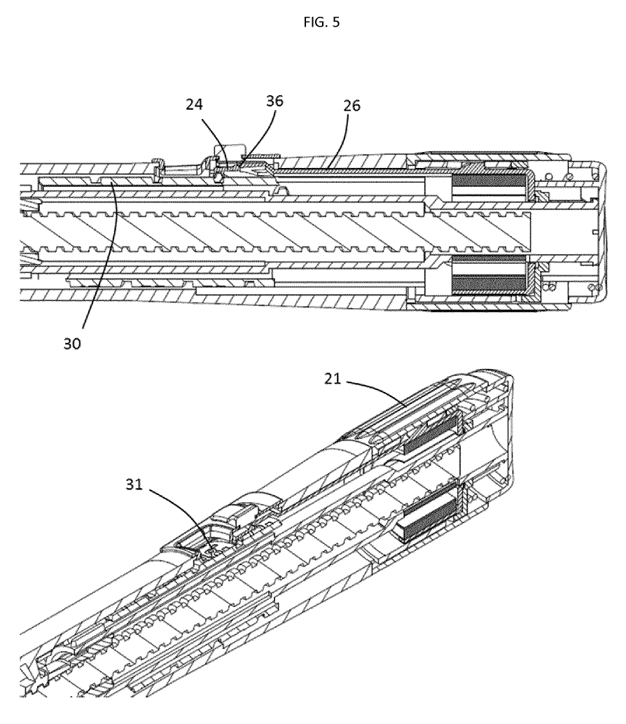

In FIG. 5 the scale drum has been turned to the value dose of “48”, and the scale drum is located towards the distal end away from the trigger button at the proximal end. The arm (24) abuts the groove in the scale drum and only applies a minimum of friction or no friction at all at the scale drum in the preparation state during dose setting, as the arm is still positioned adjacent the abutment surface (36) of the flange (26).

When altering the handheld injection device from an inactive state or during dose setting to an activated stage, the trigger button is pressed and simultaneously the flange (26) is moved towards the contact surface (27) of the arm (24). The scale drum now turns the opposite way as the torsion spring releases the stored energy. The resilient arm will pass the vertex (37) of the flange and abut the inclined contact surface (39) as illustrated in FIG. 5. The arm (24) comprises two prongs (25) which are pressed into the thread groove (32) applying the variable friction force. The user is able to adjust the velocity of the applied pressure, as the inclined contact surface (39) of the flange has more thickness near the distal end of the flange in that the inclined contact surface (39) presses the arm and the two prongs towards the scale drum to cause increased friction. When the trigger button is pressed in maximum, the contact surface of the arm is positioned such that the inclined contact surface provides most room, thus providing less pressure on the arm and decreased friction between the two prongs and the thread groove (32). This state is illustrated in FIG. 6.

In FIG. 6 a similar embodiment is illustrated as in FIG. 2. The trigger button (22) is partly pressed inwards towards the scale drum simultaneously activating the drive mechanism (not shown), releasing the spring force for dispensing, and the flange (26) is moved such that the contact surface (27) of the arm is positioned abutting the inclined contact surface (39) of the flange (26).

The resilient arm has passed the vertex of the flange and abuts approximately at the middle of the inclined contact surface (39). The two prongs (25), which are pressed into the thread groove by the flange (26), applies a variable friction due to the varying width of the thread groove, thus providing a variable counterforce, resulting in an at least more uniform dispensing force.

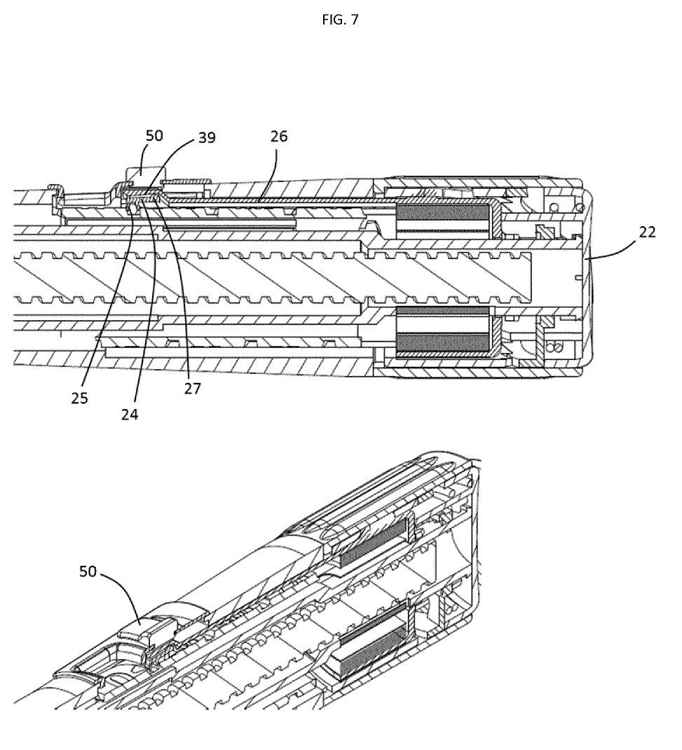

The next FIG. 7 illustrates the trigger button pressed further and the inclined contact surface (39) applies less pressure on the arm (24).

When the trigger button is pressed maximally, the contact surface of the arm is positioned where the inclined contact surface (39) provides most room and thus less pressure on the contact surface (27) of the arm. Thereby, the friction between the two prongs (25) and the thread groove (32) is decreased. Thereby, the user may be able to speed up the dispensing period during dispensing.

In FIG. 7 the handheld injection device comprises a first friction setting element (50). The first friction setting element comprises an inclined lower surface. Thus, by sliding the first friction setting element tangentially to the extent of the handheld injection device into a position having a larger thickness, the friction element will move the flange and its contact points closer to the arm and the scale drum, thereby providing more friction and thus slower delivery speed during dispensing. This may additionally allow the user to adjust the dose velocity by setting the starting level of the delivery speed determined by the trigger button (22).

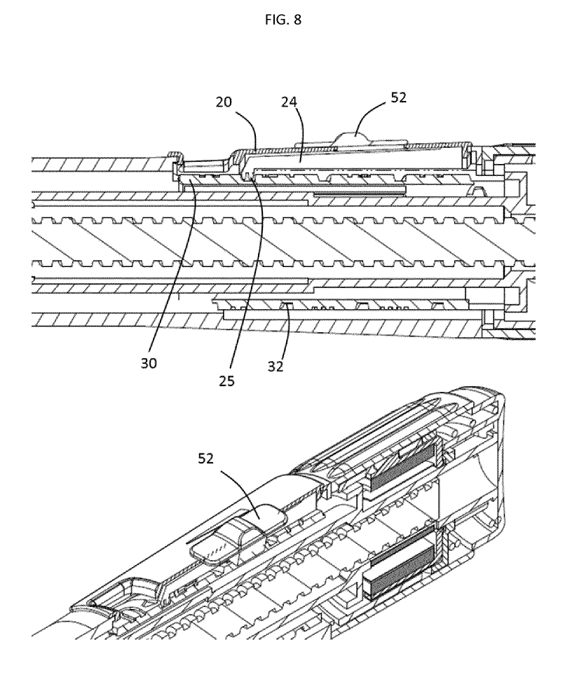

Another embodiment of the present invention is illustrated in FIG. 8. The arm (24) is fixed to a part of the outer housing (20) at a distance from the point where the two prongs (25) engage with the thread groove (32). The elongated portion of the arm provides flexibility to the arm allowing the arm to move out of engagement with the thread groove.

In the inactive state, where the dose may be set as illustrated in FIG. 8, the scale drum has been turned (clockwise) to a dose “48”. The elongated flexible arm will provide minimum of friction to the scale drum as the arm will flex and leave the thread groove (32). The outer housing comprises a void allowing the arm to move away from the engagement with the thread groove and move alongside the inner surface of the outer housing.

The embodiment illustrated in FIG. 8 comprises a second friction setting element (52) positioned movably along the elongated portion, adapted to change the distance between the attachment point of the arm and the distal end of the arm comprising the two prongs.

As the trigger button is pressed and the variable friction system is activated, the second friction setting element (52), dependent on the position, will fix the arm and determine the flexibility and thus the friction.

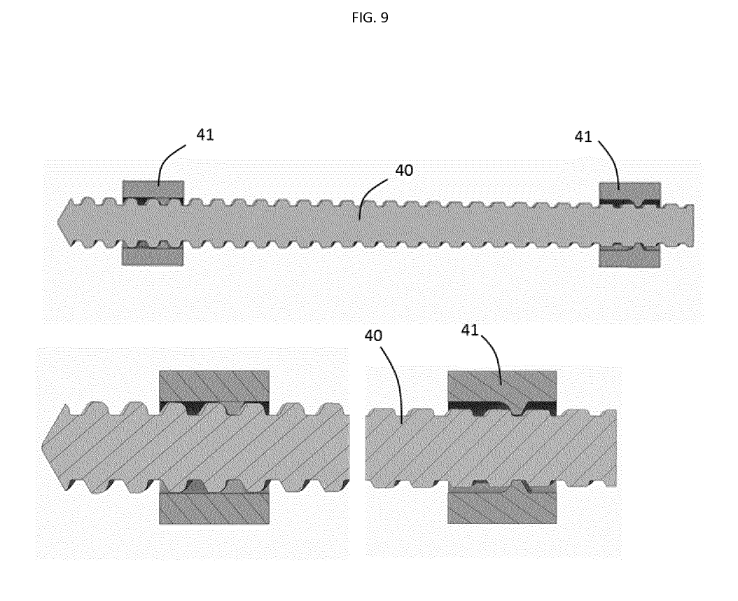

FIG. 9 illustrates another embodiment of the variable friction system.

The variable friction system comprises a piston rod (40) and one or more guiding nuts (41). The piston rod comprises a varying cross-sectional diameter. The distal end of the piston rod comprises the largest diameter, and the diameter of the piston rod is decreasing along the length of the piston rod towards the proximal end of the handheld injection device.

In the dispensing state the piston rod engages the one or more rods resulting in a friction loss. During use, as the diameter gradually decreases, the friction loss gradually reduces.

The variable friction is obtained by having most friction at the beginning of the delivery of the liquid drug and the one or more guiding nuts (41) having complementary shaped surfaces configured for gradually decreasing the friction between the piston rod and the one or more guiding nuts (41) when passing each other. Thus, the friction loss is reduced as the piston rod is moved towards the empty device state during dispensing of liquid drug.

1. A handheld injection device comprising:
a housing extending along a central axis and having a window,
a dose setting mechanism comprising a dose setting member operable to set a dose of drug to be expelled from a reservoir, and a scale drum carrying a plurality of dose indicating indicia,
a dose delivery mechanism comprising an expelling structure for expelling a set dose of drug from the reservoir, a spring member for providing energy to actuate the expelling structure, and a trigger button operable to cause energy to be released from the spring member, and
a variable friction system comprising a contact member adapted to mechanically interface with the scale drum,
wherein the scale drum is configured to move relative to the window to a dose set position in response to a dose setting operation of the dose setting member and to move from the dose set position to a dose expelled position in response to a dose expelling operation of the trigger button, and
wherein the variable friction system is configured to provide a friction force between the contact member and the scale drum which decreases gradually as the scale drum moves from the dose set position to the dose expelled position.

2. A handheld injection device according to claim 1, wherein the scale drum comprises an exterior groove having a width and a depth, wherein the contact member is configured to travel the exterior groove from a first position defined by the dose set position to a second position defined by the dose expelled position during expelling of the set dose, and wherein the contact force between the contact member and the exterior groove decreases gradually as the contact member travels from the first position to the second position.

3. A handheld injection device according to claim 2, wherein the exterior groove extends helically about the scale drum, wherein the contact member is axially fixed with respect to the housing during expelling of the set dose, and wherein the scale drum is arranged to move helically within the housing.

4. A handheld injection device according to claim 3, wherein the width of the exterior groove increases gradually from the first position to the second position.

5. A handheld injection device according to claim 3, wherein the depth of the exterior groove increases gradually from the first position to the second position.

6. A handheld injection device according to claim 2, wherein the contact member comprises two prongs arranged on a radially deflectable portion of the housing, the two prongs being adapted to engage with the exterior groove.

7. A handheld injection device according to claim 2, wherein the contact member is operatively coupled with the trigger button, and the variable friction system is configured to a) switch from a disengaged state in which the contact member is disengaged from the scale drum to an engaged state in which the contact member is engaged with the scale drum in response to the dose expelling operation of the trigger button, and b) switch from the engaged state to the disengaged state in response to a subsequent operation of the trigger button.

8. A handheld injection device according to claim 7, wherein the trigger button is capable of axial movement relative to the housing between an idle position in which the dose setting member is operable to set the dose and an activated position in which the expelling structure is actuated to expel the set dose, wherein the dose expelling operation of the trigger button comprises moving the trigger button from the idle position to the activated position, and wherein the subsequent operation of the trigger button comprises moving the trigger button from the activated position to the idle position.

9. A handheld injection device according to claim 8, wherein the trigger button is biased towards the idle position.

10. A handheld injection device according to claim 8, wherein the contact member is arranged on a radially deflectable portion of the housing, and wherein the trigger button is structurally connected to a flange, the flange comprising an abutment surface configured to interact with the radially deflectable portion of the housing and cause a radially inward deflection of the contact member towards the central axis when the trigger button is moved from the idle position to the activated position.

11. A handheld injection device according to claim 10, wherein the abutment surface comprises an inclined portion causing the radially inward deflection of the contact member to vary as a function of the axial position of the trigger button relative to the housing.

12. A handheld injection device according to claim 10, further comprising a friction setting element operable to set an initial level of the contact force.

13. A handheld injection device according to claim 12, wherein the friction setting element comprises an inclined contact surface adapted for providing a variable pressure to the flange.

14. A handheld injection device according to claim 13, wherein the friction setting element is movable in a direction tangential to a periphery of the housing.
